(19) United States
(12) Patent Application Publication
(10) Pub. No.: US 2019/0242839 A1
(43) Pub. Date: Aug. 8, 2019

(54) GAS SENSOR

(71) Applicants: TYNTEK CORPORATION, HSINCHU CITY (TW); NATIONAL CHIAO TUNG UNIVERSITY, HSINCHU (TW)

(72) Inventors: RAY-HUA HORNG, HSINCHU (TW); CHIA-CHUN YU, MIAOLI COUNTY (TW); CHUN-YI TUNG, MIAOLI COUNTY (TW); SI-HAN TSAI, HSINCHU (TW); LI-CHUNG CHENG, HSINCHU (TW)

(21) Appl. No.: 15/889,525

(22) Filed: Feb. 6, 2018

(51) Int. Cl. G01N 27/04; G01N 33/00

(52) U.S. Cl. CPC G01N 27/04; H01L 21/0242; G01N 33/0027

(57) ABSTRACT

A gas sensor is revealed, and the gas sensor comprises a sapphire substrate. An epitaxial oxide sensing layer is disposed on the sapphire substrate and formed by a thin film of single-crystalline gallium oxide series grown by using metal-organic chemical vapor deposition. The material of epitaxial oxide sensing layer include oxygen, gallium, and zinc. Two electrodes are disposed on a portion of the epitaxial oxide sensing layer. When the epitaxial oxide sensing layer senses a gas, a current will be generated and change the resistance. The two electrodes thereon receive the resistance. According to the change of the resistance, the concentration of the gas can be deduced. A heating element is further disposed below the sapphire substrate for providing the temperature required for sensing.

### FIELD OF THE INVENTION

The present invention relates generally to a sensor, and particularly to a gas sensor.

### BACKGROUND OF THE INVENTION

As social commercialization and industrialization progress, more indoor space is built, and more vehicles are adopted for meeting people's demands in leisure time, work, and commutation. Unfortunately, when people are situated in the airtight indoor space, due to incirculation of air, hazardous gas will accumulate. This will affect the living quality of people in the space. At worst, people's health will be threatened.

In general, when the indoor concentration of carbon dioxide is below 1,000 ppm, it is regarded normal and air circulation is good. When the indoor concentration of carbon dioxide is raised to 1,000 to 2,000 ppm, oxygen can be insufficient, making people feel tired and anxious. When the indoor concentration of carbon dioxide is further raised to 2,000 to 5,000 ppm, people will start to feel uncomfortable. They will have headache and feel drowsy and unfocused. Their attention will be lowered. Their heart rate will accelerate. In addition, they will feel slightly nauseated. When the indoor concentration of carbon dioxide is further raised to above 5,000 ppm, exposure to the environment can lead to serious anoxia, which will cause permanent brain injury, coma, or even death. According to realistic measurements, the concentration of carbon dioxide in the space of people's daily living can reach 2,000 to 3,000 ppm due to factors such as insufficient air exchange of air conditioners or an excessive crowd of people in the space. This concentration makes people drowsy and slightly uncomfortable. At this moment, if further control on the indoor concentration of carbon dioxide is not executed, the indoor concentration of carbon dioxide might continue to increase, which will expose people in the space in danger.

On the other hand, carbon monoxide is also a gas requiring monitoring of it concentration in people's daily lives. Carbon monoxide is a colorless and odorless chemical formed by incomplete combustion of carbon-containing materials. People may contact carbon monoxide since incomplete combustion of fuel gas or motorcycle exhaust occurs in our living environment. The affinity between carbon monoxide and hemoglobin is higher than the affinity between oxygen and hemoglobin by two to three hundred times. Thereby, when a human inhales carbon monoxide, the carbon monoxide will compete with the oxygen in the human body for the opportunity of combining with hemoglobins. Then carbon monoxide will replace oxygen and combine with hemoglobins. As a result, the oxygen concentration in human blood will be lowered. People will become unconscious and comatose gradually while they beware no abnormality. Finally, their hearts and brains will be damaged, leading to death. Given the threat of carbon monoxide toxication, early detection of increase in the concentration of carbon monoxide in airtight space is especially important.

The thin film of a general gas sensor is mostly made of tin oxide or zinc oxide with the usual growth method of thermal vapor deposition or sputtering. In the past, the gas sensors are less sensitive. In order to improve the sensitivity of sensors, the present invention provides a gas sensor having a thin film of highly crystallized gallium oxide series grown by metal-organic chemical vapor deposition for achieving preferred detection sensitivity.

### SUMMARY

An objective of the present invention is to provide a gas sensor, which adopts metal-organic chemical vapor deposition to grow thin films of highly crystallized gallium oxide series for achieving preferred detection sensitivity.

To achieve the above objective, the present invention provides a gas sensor, which comprises a substrate, an epitaxial oxide sensing layer, and two electrodes. The epitaxial oxide sensing layer is disposed on the substrate and the material of the epitaxial oxide sensing layer includes oxygen, gallium, and zinc. The two electrodes are disposed on a portion of the epitaxial oxide sensing layer. When the epitaxial oxide sensing layer senses a gas, it will generate current to change its resistance.

According to an embodiment of the present invention, the gas sensor further comprises a heating element disposed below a portion of the substrate.

According to an embodiment of the present invention, the substrate is a sapphire substrate.

According to an embodiment of the present invention, the epitaxial oxide sensing layer is a single-crystalline thin film.

According to an embodiment of the present invention, the epitaxial oxide sensing layer is a polycrystalline thin film.

According to an embodiment of the present invention, the material of the epitaxial oxide sensing layer is zinc gallium oxide (ZnGa₂O₄, ZGO).

According to an embodiment of the present invention, the application temperature of the epitaxial oxide sensing layer can reach 800° C.

According to an embodiment of the present invention, the epitaxial oxide sensing layer is annealed in nitrogen or oxygen at 800 to 950° C.

According to an embodiment of the present invention, the material of the two electrodes is selected from the group consisting of titanium/aluminum/titanium and titanium/platinum/gold.

According to an embodiment of the present invention, the gas is selected from the group consisting of carbon dioxide (CO₂), alcohol, total volatile organic compound (TVOC), and sulfur dioxide (SO₂).

According to an embodiment of the present invention, the epitaxial oxide sensing layer is used to react with the gas for generating current to raise or lower the resistance.

According to an embodiment of the present invention, the material of the heating element is selected from the group consisting of tungsten and platinum.

### BRIEF DESCRIPTION OF THE DRAWINGS

FIG. 1 shows a structure diagram of the gas sensor according to an embodiment of the present invention;

FIG. 2 shows a structure diagram of the gas sensor according to an embodiment of the present invention;

FIG. 3 shows a gas response diagrams of the gas sensor according to an embodiment of the present invention; and

FIG. 4 shows a gas response diagrams of the gas sensor according to an embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures.

In the following description, figures are used for describing various embodiments according to the present invention in detail. Nonetheless, the concept of the present invention can be embodied in many different forms, instead of being limited to the exemplary embodiments described in the present description.

To solve the problem of lower sensitivity in the gas sensor according to the prior art, the present invention provides a gas sensor formed by growing a thin film of single-crystalline gallium oxide series using metal-organic chemical vapor deposition. By using zinc gallium oxide (ZnGa₂O₄, ZGO) as the epitaxial oxide sensing layer, the sensitivity of the gas sensor can be improved effectively.

The present invention provides a gas sensor, which uses a thin film of single-crystalline gallium oxide series using metal-organic chemical vapor deposition as the epitaxial oxide sensing layer. The epitaxial oxide sensing layer is disposed on a portion of a sapphire substrate. The epitaxial oxide sensing layer reacts with the gas to generate ionized electrons for changing the resistance. According to the change of the resistance, the concentration of the gas can be deduced.

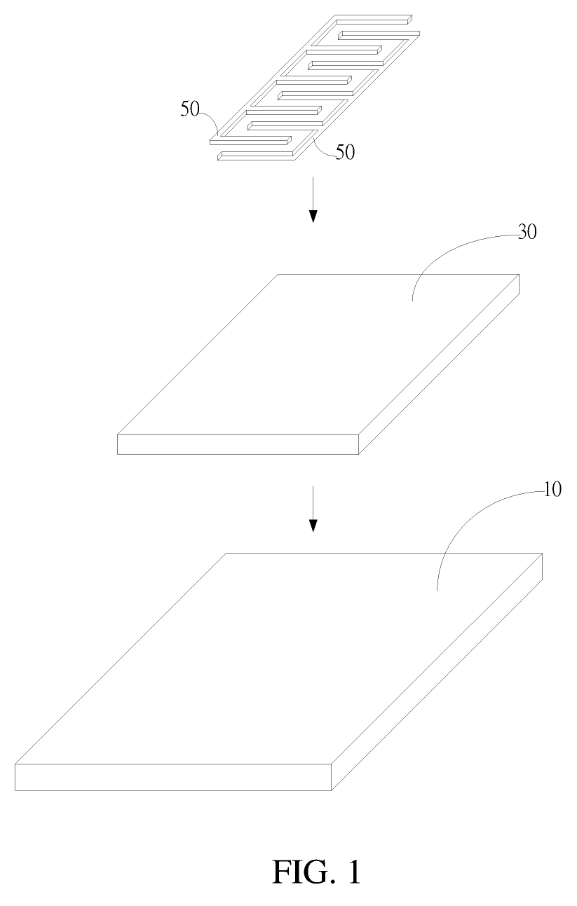

First, please refer to FIG. 1 shows a structure diagram of the gas sensor according to an embodiment of the present invention. As shown in the figure, the present invention provides a gas sensor, which comprises a substrate 10, an epitaxial oxide sensing layer 30, and two electrodes 50. The epitaxial oxide sensing layer 30 is disposed on a portion of the substrate 10. The two electrodes 50 are disposed on a portion of the epitaxial oxide sensing layer 30.

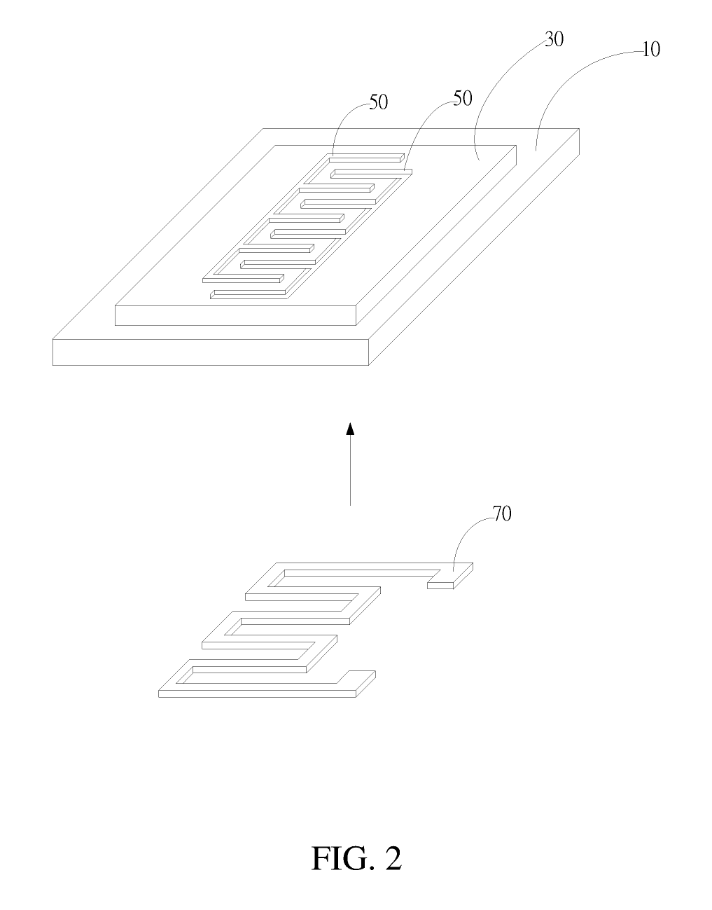

Please refer to FIG. 2, which shows a structure diagram of the gas sensor according to an embodiment of the present invention. As shown in the figure, the present invention provides a gas sensor, and further comprises a heating element 70 disposed below a portion of the substrate 10.

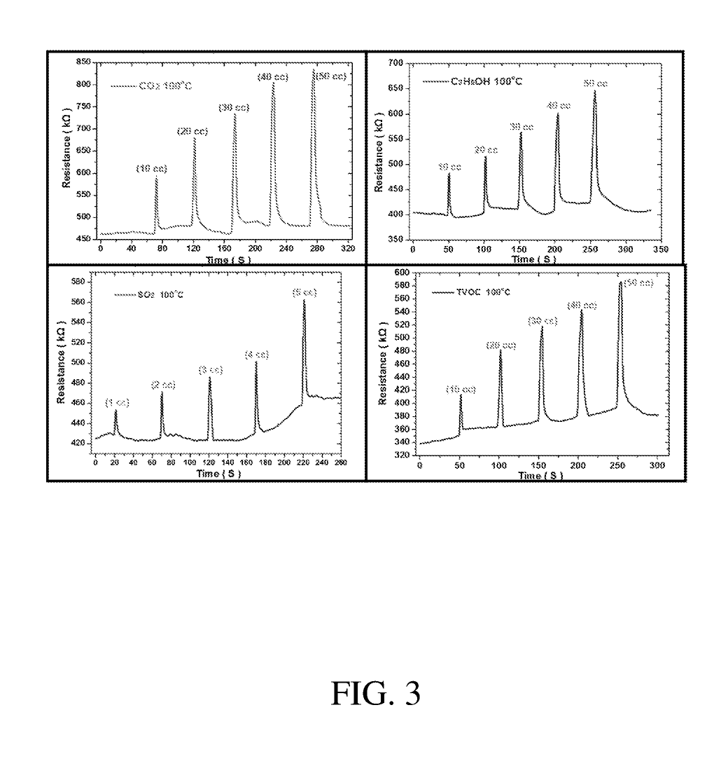

The epitaxial oxide sensing layer 30 is used for reacting with gases such as CO₂, alcohol, TVOC, and SO₂. When the gas is absorbed to the epitaxial oxide sensing layer 30, an electron will be captured, as shown in Formula 1. Next, when the temperature is increased, the sensitivity will be increased. Then the reaction rate of chemical absorption is accelerated and hence the absorption state is further stabilized, as shown in Formula 2. As the temperature reaches 100° C., oxidation reactions occur in the epitaxial oxide sensing layer 20, making the gas to lose electrons and thus raising the resistance. As shown in FIG. 3, at 100° C., the epitaxial oxide sensing layer 30 reacts with CO₂, alcohol, TVOC, and SO₂, respectively. The reactions increase the resistance. Next, in Table 1 below, the sensitivities of the epitaxial oxide sensing layer 30 on gases at 100° C. are shown. The highest sensitivity of the epitaxial oxide sensing layer 30 on CO₂ can reach 76.92%; the highest sensitivity of the epitaxial oxide sensing layer 30 on alcohol can reach 52.12%; the highest sensitivity of the epitaxial oxide sensing layer 30 on TVOC can reach 49.61%; and the highest sensitivity of the epitaxial oxide sensing layer 30 on SO₂ can reach 25.79%.

g+e⁻-→g⁻ (Formula 1)

g⁻+e⁻-→2g⁻ (Formula 2)

The symbol g shown in Formula 1 and Formula 2 represents the gas to be detected, such as CO₂, alcohol, TVOC, and SO₂.

TABLE 1

Sensitivity level

| | LV1 | LV2 | LV3 | LV4 | LV5 |
|---|---|---|---|---|---|
| CO₂ | 34.05% | 45.43% | 61.29% | 70.66% | 76.92% |
| Alcohol | 24.18% | 32.42% | 38.19% | 48.77% | 52.12% |
| TVOC | 19.22% | 36.93% | 42.03% | 43.24% | 49.61% |
| SO₂ | 7.58% | 14.82% | 17.09% | 22.53% | 25.79% |

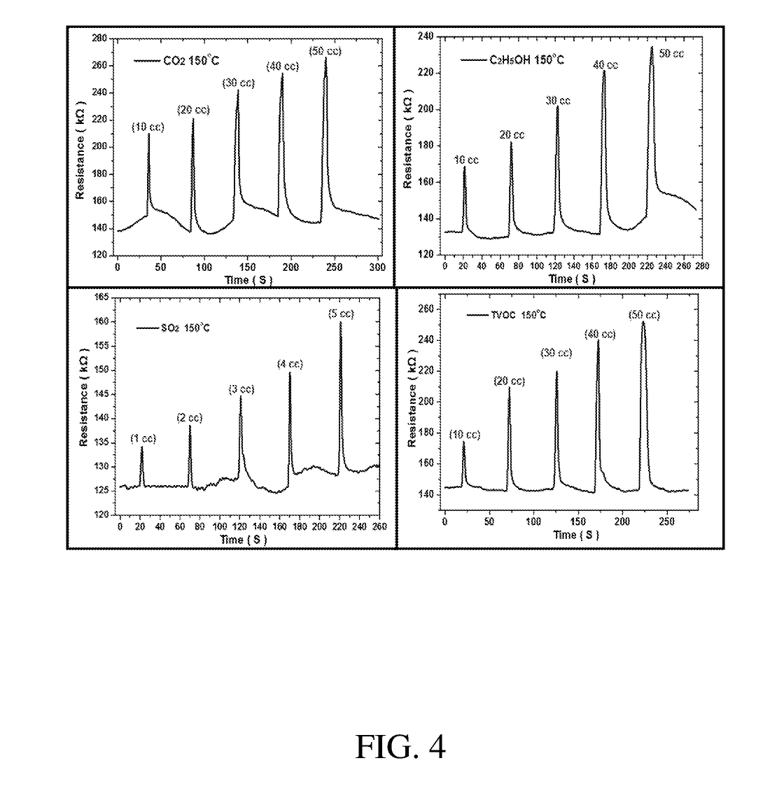

The sensitivity of the epitaxial oxide sensing layer 20 at 150° C. is superior to that at 100° C. As shown in FIG. 4, at 150° C., oxidation reactions occur in the epitaxial oxide sensing layer 30, and the epitaxial oxide sensing layer 30 reacts with CO₂, alcohol, TVOC, and SO₂, respectively. The reactions increase the resistance. Next, in Table 2 below, the sensitivities of the epitaxial oxide sensing layer 30 on gases at 150° C. are shown. The highest sensitivity of the epitaxial oxide sensing layer 30 on CO₂ can reach 84.72%; the highest sensitivity of the epitaxial oxide sensing layer 30 on alcohol can reach 70.92%; the highest sensitivity of the epitaxial oxide sensing layer 30 on TVOC can reach 78.21%; and the highest sensitivity of the epitaxial oxide sensing layer 30 on SO₂ can reach 32.71%. According to the data, it is known that the sensitivity of the epitaxial oxide sensing layer 20 at 150° C. is superior to that at 100° C.

TABLE 2

Sensitivity level

| | LV1 | LV2 | LV3 | LV4 | LV5 |
|---|---|---|---|---|---|
| CO₂ | 40.26% | 60.58% | 65.75% | 70.46% | 84.72% |
| Alcohol | 31.81% | 40.00% | 58.33% | 70.04% | 70.92% |
| TVOC | 23.69% | 51.22% | 58.29% | 77.89% | 78.21% |
| SO₂ | 7.77% | 13.76% | 16.20% | 25.97% | 32.71% |

Next, as shown in Tables 3 to 6, the detection times of the epitaxial oxide sensing layer 30 on CO₂, alcohol, TVOC, and SO₂ at 100° C. and 150° C., respectively, are all less than 10 seconds.

TABLE 3

| Response time | Response | | | | | |
|---|---|---|---|---|---|---|
| Alcohol | temperature | | 100° C. | | 150° C. | |
| (C₂H₅OH) | Rise | Fall | Rise | Fall | Rise | Fall |
| — | time | time | time | time | time | time |
| 10 cc | — | — | 2 s | 4 s | 2 s | 2 s |
| 20 cc | — | — | 2 s | 4 s | 2 s | 3 s |
| 30 cc | — | — | 3 s | 5 s | 3 s | 4 s |
| 40 cc | — | — | 5 s | 6 s | 4 s | 4 s |
| 50 cc | — | — | 6 s | 7 s | 6 s | 5 s |

TABLE 4

| | Response | | | | | |
|---|---|---|---|---|---|---|
| Response time | temperature | | 100° C. | | 150° C. | |
| CO₂ | Rise | Fall | Rise | Fall | Rise | Fall |
| — | time | time | time | time | time | time |
| 10 cc | — | — | 2 s | 3 s | 2 s | 2 s |
| 20 cc | — | — | 4 s | 5 s | 3 s | 2 s |
| 30 cc | — | — | 5 s | 6 s | 5 s | 4 s |
| 40 cc | — | — | 5 s | 7 s | 6 s | 4 s |
| 50 cc | — | — | 5 s | 7 s | 6 s | 5 s |

TABLE 5

| | Response | | | | | |
|---|---|---|---|---|---|---|
| Response time | temperature | | 100° C. | | 150° C. | |
| TVOC | Rise | Fall | Rise | Fall | Rise | Fall |
| — | time | time | time | time | time | time |
| 0.5 cc | — | — | 2 s | 1 s | 2 s | 2 s |
| 1.0 cc | — | — | 4 s | 3 s | 3 s | 2 s |
| 1.5 cc | — | — | 5 s | 4 s | 4 s | 4 s |
| 2.0 cc | — | — | 6 s | 5 s | 4 s | 3 s |
| 2.5 cc | — | — | 6 s | 6 s | 4 s | 4 s |

TABLE 6

| Response time | Room temperature | | 100° C. | | 150° C. | |
|---|---|---|---|---|---|---|
| SO₂ | Rise | Fall | Rise | Fall | Rise | Fall |
| — | time | time | time | time | time | time |
| 1 cc | 4 s | 2 s | 1 s | 3 s | 2 s | 1 s |
| 2 cc | 5 s | 3 s | 1 s | 3 s | 2 s | 2 s |
| 3 cc | 5 s | 2 s | 2 s | 3 s | 2 s | 2 s |
| 4 cc | 5 s | 2 s | 2 s | 3 s | 2 s | 2 s |
| 5 cc | 6 s | 2 s | 2 s | 4 s | 2 s | 2 s |

What is claimed is:

1. A gas sensor, comprising:
a substrate;
an epitaxial oxide sensing layer, disposed on said substrate, the material of said epitaxial oxide sensing layer including oxygen, gallium, and zinc; and
two electrodes, disposed on a portion of said epitaxial oxide sensing layer;
where said epitaxial oxide sensing layer senses a gas for generating current to change a resistance thereof.

2. The gas sensor of claim 1, and further comprising a heating element disposed below a portion of said substrate.

3. The gas sensor of claim 1, wherein said substrate is a sapphire substrate.

4. The gas sensor of claim 1, wherein said epitaxial oxide sensing layer is a single-crystalline thin film.

5. The gas sensor of claim 1, wherein said epitaxial oxide sensing layer is a polycrystalline thin film.

6. The gas sensor of claim 1, wherein the material of said epitaxial oxide sensing layer is zinc gallium oxide (ZnGa₂O₄, ZGO).

7. The gas sensor of claim 1, wherein the application temperature of said epitaxial oxide sensing layer reaches 800° C.

8. The gas sensor of claim 1, wherein said epitaxial oxide sensing layer is annealed in nitrogen or oxygen at 800° C. to 950° C.

9. The gas sensor of claim 1, wherein the material of said two electrodes is selected from the group consisting of titanium/aluminum/titanium and titanium/platinum/gold.

10. The gas sensor of claim 1, wherein said gas is selected from the group consisting of carbon dioxide (CO₂), alcohol, total volatile organic compound (TVOC), and sulfur dioxide (SO₂).

11. The gas sensor of claim 1, wherein said epitaxial oxide sensing layer reacts with said gas to generate current for increasing or decreasing the resistance.

12. The gas sensor of claim 2, wherein the material of said heating element is selected from the group consisting of tungsten and platinum.
